(19) United States
(12) Patent Application Publication
(10) Pub. No.: US 2004/0081623 A1
(43) Pub. Date: Apr. 29, 2004

(54) Perfusion imaging method

(76) Inventors: Morten Eriksen, Oslo (NO); Henrik Rasmussen, Oslo (NO)

Correspondence Address: AMERSHAM HEALTH, IP DEPARTMENT, 101 CARNEGIE CENTER, PRINCETON, NJ, 08540-6231, US

(21) Appl. No.: 10/466,055

(22) PCT Filed: Jan. 11, 2002
(86) PCT No.: PCT/NO02/00015

(30) Foreign Application Priority Data
Jan. 12, 2001 (NO) 20010234

(51) Int. Cl. A61K 49/00

(52) U.S. Cl. 424/9.52

(57) ABSTRACT

This invention relates to ultrasound imaging, more specifically to a method of ultrasound investigation involving the use of an ultrasound contrast agent and administration of at least two gases or gas mixtures having different partial pressure of inert gas. The method may be used in assessing blood perfusion in tissue. A timed change of administered gases gives a subsequent change in contrast echogenicity. The gases are preferably administered by inhalation. The invention further relates to gas-microbubble containing ultrasound contrast agents used according to the method, and to kits to be used in assessments of tissue perfusion.

This invention relates to ultrasound imaging, more specifically to a method of ultrasound investigation involving the use of an ultrasound contrast agent and administration of at least two gases or gas mixtures having different partial pressure of inert gas. The method may be used in assessing blood perfusion in tissue. A timed change of administered gases gives a subsequent change in contrast echogenicity.

It is well known that contrast agents comprising gas microbubbles are particularly efficient backscatterers of ultrasound due to low density and ease of compressibility of the microbubbles. Such microbubbles, if stable to maintain a sufficient size in vivo, may permit highly effective ultrasound visualisation of tissue microvasculature, for example in the myocardium. Stability of the microbubbles may reside in characteristics of the gas, for example a low water solubility, or use of any stabilising material, for example an encapsulating surfactant. The size of microbubbles is of importance since their echogenic intensities increase with size.

The use of ultrasonography to measure blood perfusion, i.e. blood flow per unit of tissue mass, is of potential value in studies of hypoperfused tissue, e.g. to detect and characterise stenoses, and in detection or characterisation of hyperperfused tissue, for example tumours, which typically have deviating, often higher, vascularity than healthy tissue. Methods allowing differentiation between hyperperfused, hypoperfused and normally perfused tissue are thus of high diagnostic value. In this field, a number of gas-containing contrast agents have shown promising potential, e.g. the marketed Optison™ (Mallinckrodt) comprising protein-encapsulated microbubbles, and Sonazoid™ (Nycomed Imaging), an agent in development comprising phospholipid-stabilised microbubbles.

Continued attempts have been made to obtain ultrasound images with clear boundaries of neighbouring regions differing in perfusion, to characterise perfusion at the local level. Usually, ultrasound imaging of cardiac perfusion is dependent on the use of a contrast agent. With techniques currently available only perfusion differences between relatively larger neighbouring regions of the heart, not between minor areas, can be demonstrated.

One route for such attempts to discriminate perfusion between neighbouring areas is to image flow of contrast agent particles through smaller regions. This will, however, only indicate the average perfusion along the transit route of the contrast agent, thus obliterating differences along the route as well as between closely neighbouring areas. The actual site e.g. of stenoses causing reduced perfusion may thus not easily be located. WO98/47533 (Nycomed Imaging) discloses a method of measuring tissue perfusion comprising administering an ultrasound contrast agent, irradiating a target tissue with at least one pulse of ultrasound to destroy or modify the echogenic properties of the contrast agent in the target region, and ultrasonically detecting and quantifying the rate of flow of either further contrast agent into said target region or modified contrast agent out of said target region.

Another route is to deposit contrast agent microbubbles in the tissue, whereby the number of deposited microbubbles may reflect the degree of perfusion. One such method is described in WO98/17324 and WO99/53963 (both of Nycomed Imaging), whereby microbubbles after passage of the pulmonary system are caused by application of ultrasound to grow at least transiently by fusion with emulsion droplets containing a co-administered volatile agent. When grown to sizes not allowing further passage in the microvasculature, the deposited microbubbles can report on perfusion by their concentration in the tissue and thus by their echo intensity.

Use of an inhalable contrast agent, comprising a mixture of at least 20% oxygen with water insoluble perfluorocarbons, is disclosed in WO96/40288 (Mallinkrodt). The inhalable contrast agent forms microbubbles in vivo after having passed the lungs.

Utility of specific inhalation gases for improving contrast agent echogenicity was disclosed in WO98/03205 (Quay), wherein a specific gas or gas mixture is inhaled to counteract degassing of simultaneously administered microbubbles during their passage of the lungs. Inhalation gases include C1 to C10 fluorocarbons, preferably corresponding to gases of the microbubbles administered.

The efficient gas exchange in the lungs are utilised in both of WO96/40288 and WO98/03205. The purpose with gas inhalation in these publications is to create bubbles or to maintain their size. Neither of these disclosures take any advantage of changing the inhalation gas as part of the procedure in order to create a dynamic response in the resulting echo signal.

Though several approaches have been suggested for assessing perfusion e.g. in myocardium, there is still a continued need to provide contrast agents and methods that can reliably trace moderate or minor degrees of perfusion differences, especially in myocardial tissue. An inherent accuracy limitation of echogenic intensity measurements makes it desirable to have contrast agents and imaging procedures which can convert intensity ratios of ultrasound echo signals between adjacent tissue regions into enhanced or amplified echo values, beyond the underlying perfusion ratios.

It has now surprisingly been found that by administering specific gases, and particularly by changing the administered gas as part of the imaging procedure, the size and echogenicity of administered ultrasound contrast agents can be changed in a way to reflect tissue perfusion. Thus, ultrasound contrast agents may be formulated to contain gas microbubbles being capable of developing a change in echogenicity upon contact with tissue having a partial pressure of inert gas different from the partial pressure of inert gas inside said microbubbles. Such microbubbles are useful in a method of assessing tissue perfusion involving a timed changing of partial pressure of inert gas of the administered gas.

The primary observation upon which the current invention is based, is that the partial pressure of oxygen in the myocardium remains practically unchanged by a change in inhaled oxygen fraction. The myocardial partial pressure of oxygen is mainly determined by the shape of the haemoglobin oxygen disassociation curve, the perfusion of blood through the tissue, and the amount of oxygen consumed by the tissue, and not so much by the oxygen partial pressure in arterial blood. As an example, breathing of pure oxygen will generate a sum of partial pressures of dissolved gases in myocardial tissue of about 18 kPa, where oxygen contributes with about 4 kPa, CO₂ with 6.4 kPa, H₂O with 6.3 kPa. The corresponding sum of partial pressures when breathing room air containing 78% N₂ is about 91 kPa, mainly caused by the presence of dissolved N₂ in the tissue.

The change in partial pressure of inert gas in an inhalation gas will subsequently develop a change in tissue partial pressure of inert gas, first developing in tissue regions with a high perfusion, and later in regions with low perfusion. If we assume that the solubility of the inert gas in blood and tissue are similar, and that diffusion equilibrium between tissue and capillaries is continuous, then the time course of inert gas partial pressure will be an exponential function with a time constant that is determined only by the perfusion of the tissue. If the partial pressure of inert gas in arterial blood supplying a tissue with a perfusion of \dot{Q} is changed from p₀ to p₁ at t=0, then the time course of partial pressure of inert gas in the tissue as a function of time after the change will be:

p(t)=p₁+(p₀−p₁)exp(−t\dot{Q}) eq. 1)

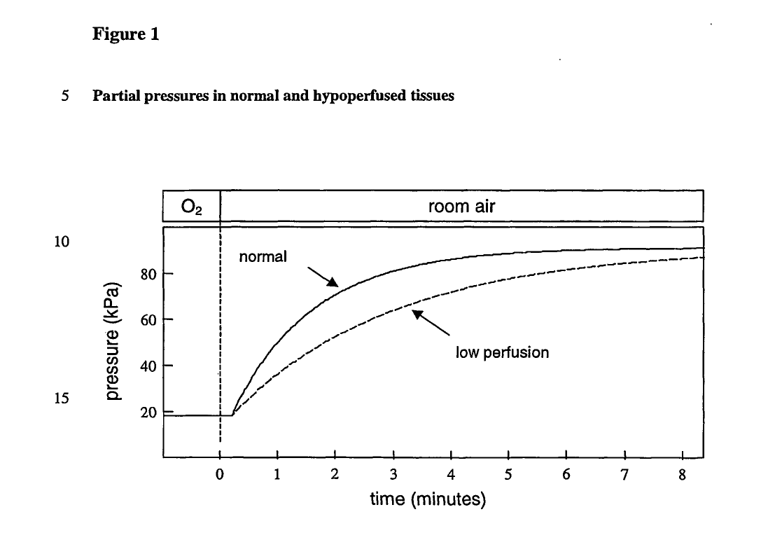

A typical value of perfusion for normal myocardium is 70 ml/(min * 100 ml)=0.012 s⁻¹. By inserting this into eq. 1), we find that when switching between two arterial gas partial pressures, the exponential change in tissue partial pressure will be halfway completed after about 59 s. If the perfusion is reduced to half of its normal value, this time will be 118 s. Thus at selected time points, there will be a considerable difference in gas partial pressures in normal and hypoperfused tissues. This is illustrated in FIG. 1, where the time course of the summed partial pressure of all gases in the myocardium is plotted against time for normally perfused tissue (solid line), and for a tissue with 50% of the normal perfusion (dotted line). A slight delay (10 s) is indicated between the change in inhaled gas and the onset of changes in the tissue, this is caused by the time needed to exchange the gas already contained in the airways, and the bloodstream transport delay between the lungs and the coronary circulation.

If the changes are subsequently reflected in differences in microbubble echogenicity, as happens when bubble size change as a consequence of gases diffusing between the bubble interior and the surrounding tissue or blood, the degree of perfusion of areas or regions will become apparent in characteristic temporal changes in echo signal pattern. By adequate choice of contrast agents, gases to be administered, and timing of administrations of said gases and contrast agent, useful assessments of perfusion of tissue areas or regions can be obtained.

Thus, according to a first embodiment of the invention there is provided a method of ultrasound investigation of a human or non-human animal subject comprising

i) administering a gas-microbubble containing ultrasound contrast agent to said subject

ii) administering at least two gases or gas mixtures to said subject, said gases or gas mixtures having different partial pressure of inert gas, said gases or gas mixtures being administered either prior to, during and/or after the administration of said ultrasound contrast agent

iii) detecting ultrasound signals from said subject

iv) optionally, generating an image from said detected signals

The method may be used in assessments of tissue perfusion. By analysing the generated image and recorded echo signals the degree of blood perfusion in tissue may be assessed.

The gases or gas mixtures being administered are preferably administered by inhalation.

Viewed from a further aspect the method of the invention has a timed changing from a first administered gas to a second administered gas with a different partial pressure of inert gas, wherein for both the first and the second inhalation gases, the term “gas” covers single gases or gas mixtures. The gases may conveniently be administered by inhalation. The mixtures might contain different amounts of oxygen, ranging from 15% to 100%, and their inert gas components might differ in composition. A change in partial pressures of inert gas during administration causes an immediate and corresponding change in partial pressure of inert gas in tissues. When, according to the invention, the transit time of the microbubbles through the tissue is sufficiently high to allow a sufficient exchange of gases between microbubbles and tissues to take place, and when the microbubbles have the capability of changing echogenicity thereby, for example by changing size, then a change in echogenicity of the microbubbles may reflect a change in tissue partial inert gas pressure.

Viewed from yet another aspect the invention provides the use of a gas-microbubble containing ultrasound contrast agent and at least two gases for the manufacturing of an ultrasound imaging agent for detecting ultrasound signals from a subject. The ultrasound imaging agent comprises an ultrasound contrast agent, comprising gas-microbubbles, and at least two gases or gas mixtures. The gases or gas mixtures have different partial pressure of inert gas and is administered, preferably by inhalation. The ultrasound imaging agent may preferably be used in assessing the degree of perfusion in tissues.

The gas or gas mixture being administered, normally inhaled, may be a single gas, which in normal instances is oxygen, or a mixture of several gases, whereof at least one is an inert gas. The inert gas will most commonly be nitrogen, but can be any gas or mixture of gases being metabolically inert and biocompatible. Preferred gases are nitrogen, helium, argon, other noble gases, N₂O, or mixtures thereof. As an example, all or part of the nitrogen may be replaced e.g. by helium, providing essentially the same effect of tissue gas changes, microbubble size change and amended echogenicity. Nitrogen is a most preferred inert gas.

As part of the invention the administered gases have different partial pressure of inert gas. Viewed from a further aspect of the invention the method preferably comprises the administration of two gases, a first gas and a second gas wherein administration of the first gas or gas mixture is followed by administration of a second gas or gas mixture. Said second gas or gas mixture has a low partial pressure of inert gas when said first gas or gas mixture has a high pressure of inert gas, and it has a high pressure of inert gas when said first gas or gas mixture has a low pressure of inert gas.

By a high partial pressure of inert gas is meant herein that the partial pressure of inert gas is between 75 kPa and 85 kPa, preferably being about 79 kPa, for example as for nitrogen and argon in room air. By a low partial pressure of inert gas is herein meant that the partial pressure of inert gas is below 60 kPa, preferably below 40 kPa, more preferably below 20 kPa and most preferably below 5 kPa, e.g. as for oxygen or oxygen-rich gas mixtures of common use in medicine. It is to be understood that the balance will normally contain oxygen with partial pressures at least sufficient for the subject not to suffer from oxygen deficiency.

During inhalation, nitrogen and other inert gases will be present in all tissues at approximately the same partial pressure as in the gas being inhaled (with a delay when the gas is changed). This is in contrast to the behaviour of tissue partial pressures of oxygen, where, unless the oxygen concentration in the inhalation gas is decreased blow the normal level of 20-21 kPas, tissue partial pressures of oxygen change insignificantly upon changes in the inhalation gas, due to oxygen binding capacity of haemoglobin. Thus, one may, by changing partial pressure of inert gas in the inhalation gas, be able to control the local partial pressure of inert gas in tissues, without causing any substantial change in the tissue partial pressures of gases that are essential for metabolism such as O₂ and CO₂. As an example, breathing of 100% oxygen will cause a substantial decrease in the total gas saturation in tissues such as the myocardium.

Thus, for example, when inhalation of a first gas with a low partial pressure of inert gas, for a period of time sufficient to deplete the tissues of inert gas, is changed to a second gas with a high partial pressure of inert gas, the change will subsequently cause an increase in tissue partial pressure of inert gas. This so-called inert gas “wash-in” of tissues is due to gas exchange with the perfusing blood having a high partial pressure of inert gas. In highly perfused and homogenous organs, such as the myocardium with its high capillary densities and uniform composition, inert gas exchange occurs rapidly due to short diffusion distances and the time constant of the inert gas exchange is inversely related to tissue blood perfusion. For normally perfused myocardium, the time constant of nitrogen exchange is somewhat more than one minute.

When inhalation of a first gas with a high partial pressure of inert gas is changed to a second gas with a low partial pressure of inert gas for a period of time sufficient to equilibrate the tissue of inert gas, the change will subsequently cause a decrease in tissue partial pressure of inert gas. This is a so-called “wash-out” procedure as the inert gas with high pressure is “washed” out, or removed, from the tissue. The transport of inert gas from the tissue is by rapid diffusion into the capillary network and then by convective transport by the venous bloodstream, the latter being highly dependent on the amount of blood perfusing the tissue.

A wash-in procedure wherein inhalation of a first gas with a low partial pressure of an inert gas is changed to inhalation of a second gas with a high partial pressure of an inert gas is a preferred embodiment of the invention.

The microbubbles used in the invention need to be sufficiently stable in vivo to provide an echogenic signal. Any biocompatible gas may be contained in the microbubbles, the term “gas” as used herein including any substances (including mixtures) at least partially being in gaseous or vapour form at the normal human body temperature of 37° C. Bubbles that contain gas components with boiling points below body temperature are of particular interest, since an outward diffusion of inert gas (typically N₂) from such a bubble into an undersaturated environment will cause the remaining gas inside the bubble to condense into a fluid, thus converting the bubble into a non-echogenic fluid droplet. The stability of the microbubbles will at least partly reside in characteristics of the gas, e.g. such as a low water solubility. Thus, preferred gases are of low water solubility e.g. such as fluorinated gases, for example fluorocarbons or sulfur fluorides such as sulfur hexafluoride; perfluorocarbons such as perfluoropropane, perfluorobutanes, perfluoropentanes and perfluorohexanes are most preferred. These gases are particularly advantageous due to the high stability in the bloodstream of microbubbles containing such gases.

Several types of gas microbubble-containing ultrasound contrast agents can be formulated to quickly and preferably reversibly change size, and accordingly echogenicity, of the microbubbles upon contact with tissues with a different partial pressure of inert gas. For microbubbles of conventional free-flowing ultrasound contrast agents, transit time through myocardial tissue will usually be in the order of 10 seconds. Preferably the transit time can be prolonged by any mechanism or formulation, to allow for efficient gas exchange between tissue and microbubbles, with corresponding changing of microbubble size upon exchange with tissue inert gas.

A sudden change in inhaled gas from one containing a high fraction of inert gas, such as room air, to one with a low content of inert gas, such as 100% O₂, will cause a wash-out of N₂ from the tissues in a time period of a few minutes. In a time interval, typically 1-2 minutes after the change in inhaled gas, there will be an appreciable difference in the content of inert gas between normally perfused tissue and hypoperfused tissue, with the hypoperfused tissue containing the highest concentration of inert gas. The hypoperfused tissue will in this situation provide a more favourable environment for bubbles to be echogenic. In the same situation, the bubbles that are injected into the bloodstream will be distributed by number between the normal and hypoperfused tissue in a manner that cause a higher concentration of bubbles in the normally perfused tissue. Thus, the effects of tissue gas tensions and bubble distribution on the difference in overall echo intensity between the different tissue regions will be in opposite directions, and the diagnostic utility will be low. On the other hand, using a change in inhaled gases from one with a low content of inert gas, such as 100% O₂, to a gas with a high content of inert gas, such as room air, will give a condition of a high content of inert gas in normally perfused tissue, and a low content of inert gas in hypoperfused tissue some 1-2 minutes after the change in inhaled gas composition. The effects of bubble number and tissue gas tensions will in this situation be synergistic, and will result in a highly desired amplification of the difference in overall echo intensity between normal and hypoperfused tissue regions.

An especially preferred embodiment of the invention is a method using a wash-in procedure wherein the first gas has a high content of oxygen and a low content of an inert gas, and wherein the second gas has a high content of inert gas. Most preferably the first gas comprises oxygen, preferably 100% oxygen, and the second gas comprises room air.

Thus, microbubbles having a prolonged transit time through tissues, e.g. at the order of 1 minute or more, are preferred for utilisation in the method according to the invention. Although microbubbles with such prolonged transit times are for simplicity termed as “deposited”, there is no need, or even no wish, that microbubbles be permanently deposited; they should rather be temporarily deposited with transit times sufficient for microbubbles to exchange gases with the surrounding tissue.

After the imaging procedure has been terminated, microbubbles should preferably be easily disposed of and eliminated from the body. It is an advantage of the method according to the invention that if wanted, one may at the end select an inhalation gas that will cause the microbubbles to obtain a reduced size, which will facilitate their disposal.

The preferred prolonged transit time for microbubbles through tissues may be achieved in a number of ways. A first class of stabilised gas microbubble-containing ultrasound contrast agents useful according to the invention is disclosed in WO98/17324. A combined preparation comprises a stabilised dispersed gas and a co-administered composition comprising a volatile component capable of evaporation in vivo into the dispersed gas so as at least transiently to increase the size of the microbubbles. Ultrasound may be applied to promote growth of said microbubbles, and the grown microbubbles may then be transiently deposited in capillaries e.g. of the myocardium. This gives the advantageous prolonged contact times of microbubbles with tissues, facilitating inert gas exchange with said tissues resulting in changes in microbubble size and echogenicity.

A second class of gas microbubble-containing ultrasound contrast agents useful according to the invention is disclosed in WO-A-9416739 (Sonus); the microbubbles similarly have depositing capabilities related to microbubble growth in vivo, due to an expansion of a phase shift agent undergoing a phase shift in vivo caused e.g. by the increased temperature of the human body, or by chemical and/or physical factors such as locally applied ultrasound etc.

Microbubbles of the first and second class described above, being capable to increase in size after passage through the pulmonary system, represent a special advantage as they may initially be designed to be small enough, e.g. 7-10 micrometer or less, to pass the pulmonary capillaries before increasing in size. Also microbubbles being initially larger than 7-10 micrometer can pass the pulmonary system when they contain a mixture of one or more relatively blood-soluble or otherwise outwards diffusible gases such as air, oxygen, nitrogen or carbon dioxide together with one or more substantially insoluble and non-diffusible gases such as perfluorocarbons.

More particularly, as viewed from a further aspect the invention provides the use of a gas microbubble-containing ultrasound contrast agent and at least two gases in the manufacture of a ultrasound imaging agent, wherein said contrast agent is a combined preparation for simultaneous, separate or sequential use as a contrast agent in ultrasound imaging, said preparation comprising:

i) a first composition which is an injectable aqueous medium comprising dispersed gas microbubbles; and

ii) a second composition which is an injectable oil-in-water emulsion wherein the oil phase comprises a volatile component capable of evaporation in vivo into said dispersed gas microbubbles so as at least transiently to increase the size thereof.

Said compositions may further comprise material serving to stabilise said dispersed gas microbubbles and said emulsion. Preferably said materials are present at the surfaces of the dispersed gas microbubbles and the droplets of the dispersed oil phase emulsion which have affinity for each other, preferreably said surface materials have opposite charges.

Still a further class of gas microbubble-containing ultrasound contrast agents useful according to the invention is disclosed in WO98/18500 and WO98/18501 (both of Nycomed Imaging). The contrast agents have depositing capabilities related to tissue-specific vectors located on the surface material of stabilised microbubbles, said vectors having affinity e.g. to receptors of a tissue, such as aberrant myocardial tissue.

Besides choice of contrast agents and gases to be administrated, controlled timing of the events of administering of contrast agent and gases are important according to the invention. For certain contrast agents and gases chosen, the timing of administering gases, especially the time for changing between said gases, may need to be adjusted; this may be done by simple experimentation. Time windows indicated hereinbelow should be regarded as indications, rather than values strictly being adhered to. It may especially be noted that wash-in and wash-out variations of the method may require different timing of events.

A first time period of significance is the duration of administration of a first gas or gas mixture, which time period needs to be sufficient to allow for at least substantial equilibration of tissues with the first gas. In practice a period of at least 5 minutes, preferably at least 10 minutes has been found to satisfy this requirement. When speaking about duration of administration here, this would normally refer to the duration of inhalation.

A second timing parameter is the time point of change from the first to the second gas, with reference to start of administering of the contrast agent. This time point will be different for wash-in and for wash-out variations of the method, and it will be different dependent on the administration method of the contrast agent, e.g. administration as a bolus and/or infusion.

Thus, for a typical wash-in embodiment according to the invention, the change from the first gas (low partial pressure of inert gas) to the second gas (high in inert gas) should preferably take place before administering of the contrast agent is started, e.g. 90 to 0 seconds before, preferred 60 to 15 seconds before, and most preferred about 30 seconds before start of administering of the contrast agent.

When the wash-in embodiment is comprising infusion of the contrast agent, the change from the first gas (low in inert gas) to the second gas (high in inert gas) should typically take place not more than 90 seconds before administering of the contrast agent is started, preferably not more than 60 seconds before, and most preferably not more than 30 seconds before start of administering of the contrast agent; the gas change is to be performed no later than about 10 seconds after administering of contrast agent is started.

A wash-in infusion embodiment according to the invention may beneficially be used with a “deposit” type of ultrasound contrast agent by combining a probing, low-dose infusion with a bolus of the same contrast agent at the end of the infusion, preferably administered when echogenicity differences become apparent in the tissue; alternatively the infusion rate may at this time be substantially increased. This will enable administration of the majority of the contrast agent dose at the point in time most relevant in the individual, without prior knowledge of this time. More generally speaking, in a method according to the invention the contrast agent may be administered either as an infusion and/or as a bolus. In some instances an infusion of the contrast agent may beneficially be followed by a bolus injection.

For a typical wash-out embodiment according to the invention, the change from the first gas (high partial pressure of inert gas) to the second gas (low partial pressure of inert gas) may preferably take place after administering of the contrast agent is started, e.g. 0 to 120 seconds after, preferably 15 to 90 seconds after, and most preferably about 30 seconds after start of administering of the contrast agent.

In performing the imaging according to the invention, a contrast agent as described is administered by any suitable route, for example by intravenous injection. The administration must be performed in a controllably timed way with regard to the time of changing of administered gas, as described above. For example, a contrast agent of the preferred class, such as a combined preparation as disclosed in WO98/17324, may be injected shortly before changing of the inhalation gas. This allows the contrast agent to pass the lungs, the microbubbles to grow e.g. after application of localised ultrasound as disclosed in WO98/17324, and further to become deposited in myocardial microvessels. Using such wash-out procedure, by changing the inhaled gas from for example room air to 100% oxygen, we have found that the contrast agent may advantageously be injected some 60 to 120 seconds, preferably about 90 seconds, prior to changing of inhalation gas in an open chest dog model. More preferably a wash-in procedure is used. For example, a contrast agent of the preferred class, such as a combined preparation as disclosed in WO98/17324, may be injected shortly after changing of the inhalation gas. This allows the contrast agent to pass the lungs, the microbubbles to grow e.g. after application of localised ultrasound as disclosed in WO98/17324, and further to become deposited in myocardial microvessels. Using such wash-in procedure we have found that the contrast agent may advantageously be injected some 0-90 seconds, preferably about 30 sec, after changing of inhalation gas in an open chest dog model. The wash-in procedure gives the added advantage of a synergistic effect on the difference in tissue echo intensity between normally perfused and hypo-perfused regions, since both the numerical distribution of microbubbles between normal and under-perfused regions, and the effects of tissue gas tensions will contribute to the echo intensity difference in the same direction. The adjustment of these times to be appropriate for a human patient will not need extensive experimentation.

Microbubbles may preferably be stabilised by gas-stabilising material e.g. by being at least partially encapsulated. This stabilising material may, for example, comprise a coalescence-resistant surface membrane such as a filmogenic protein, a polymer material, e.g. such as polylactic acid, polyglycolic acid, or copolymers of polylactic and polyglycolic acid, a non-polymeric and non-polymerisable wall-forming material, or a surfactant, such as one or more phospholipids.

Phospholipid-containing stabilisers are preferably employed in accordance with the invention, and representative examples of useful phospholipids include lecithins; phosphatidic acids; phosphatidylethanolamines; phosphatidylserines; phosphatidylglycerols; phosphatidylinositols; cardiolipins; sphingomyelins; mixtures of any of the foregoing and mixtures with other lipids such as cholesterol. Negatively charged phospholipids such as phosphatidylserines, phosphatidylglycerols, phosphatidylinositols, phosphatidic acids and/or cardiolipins are particularly advantageous.

A variety of ultrasound techniques may be employed in a method according to the invention, such as B-mode-based or Doppler-based (including decorrelation) imaging methods, both including linear and non-linear imaging methods.

Yet a further aspect of the invention is a kit comprising a gas-microbubble containing ultraound contrast agent and at least two gases or gas mixtures having different partial pressure of inert gas. Similarly, an aspect of the invention is an ultrasound imaging agent comprising a gas-microbubble containing ultrasound contrast agent and at least two gases or gas mixtures having different partial pressure of inert gas. Such kit or ultrasound imaging agent may be used in evaluations of the degree of perfusion of tissues, i.e. assessing whether tissue is hypoperfused, hyperperfused or normally perfused.

The method according to the invention is particularly applicable to highly vascularised tissue being homogeneous in structure and with a low content of lipid, providing rapid and efficient blood distribution to and gas exchange with the tissue. A preferred type of tissue is myocardium, which is very well vascularised.

Tumours may be visualised by the method according to the invention, either as hyperperfused or hypoperfused regions, or as more composite regions having for example a necrotic and hypoperfused core and normally perfused or hyperperfused outer parts.

For stenotic arteries, the region supplied by these arteries may be hypoperfused due to a reduced flow, and thus such regions may be studied by a method according to the invention. However, the reduction in blood flow in tissue supplied by a stenotic artery may become less evident due to an inherent autoregulation counteracting the reduced flow, usually by dilatation of the vessels. To differentiate stenotic regions from normal tissue one may employ the well known technique of applying physical or pharmacological stress, e.g. by administering a vasodilator to increase flow in normal vessels, whereas the already maximally dilated arterioles supplied by the stenoic vessels are substantially unable to increase their flow.

Applying stress, for example by administering of a vasodilator, may be used in conjunction with the method according to the invention; the vasodilator may be applied before, during or after administration of contrast agent and change of inhalation gas mixtures.

Representative vasodilator drugs useful in combination with a method in accordance with the invention include endogenous/metabolic vasodilators, such as adenosine; sympathetic activity inhibitors; smooth muscle relaxants; beta receptor agonists, such as dobutamine; alpha receptor antagonists; organic nitrates; angiotensin converting enzyme (ACE) inhibitors; angiotensin II antagonists (or AT1 receptor antagonists); calcium channel blockers; and endothelium-dependent vasodilators.

The following examples serve to illustrate the invention.

Preparation 1

Perfluorobutane Gas Dispersion with Negatively Charged Surface Material

Hydrogenated phosphatidylserine (5 mg/ml in a 1% w/w solution of propylene glycol in purified water) and perfluorobutane gas were homogenised in-line at 6800 rpm and ca. 40° C. to yield a creamy-white microbubble dispersion. The dispersion was fractionated to substantially remove undersized microbubbles (<2 μm) and the volume of the dispersion was adjusted to the desired microbubble concentration by adding aqueous sucrose to give a sucrose concentration of 92 mg/ml. 2 ml portions of the resulting dispersion were filled into 10 ml flat-bottomed vials specially designed for lyophilisation, and the contents were lyophilised to give a white porous cake. The lyophilisation chamber was then filled with perfluorobutane and the vials were sealed. Prior to use, water was added to the vials and the contents were gently hand-shaken for several seconds to give a perfluorobutane microbubble dispersion; the concentration of microbubbles in the dispersion was 1.1% v/v and the median microbubble size was 2.7 μm.

The negatively charged perfluorobutane gas dispersion is administrated intravenously in amounts corresponding to 0.1 μl gas/kg body weight.

Preparation 2

Perfluoromethylcyclopentane Emulsion with Positively Charged Surface Material

Stearylamine (25 mg) and distearoylphosphatidylcholine (477 mg) were placed in a 250 ml round bottom flask and chloroform (25 ml) was added. The flask was put on a rotavapor and the chloroform was removed by evaporation at 350 mbar using a bath temperature of 45° C. In order to remove residual traces of solvent the sample was exposed to ca. 20 mbar vacuum overnight. Thereafter, a buffer solution of 10 mM Tris (100 ml) was added and the flask was rotated at full speed for 10 minutes while immersed into a 80° C. water bath. The sample was cooled to room temperature overnight before placed in a refrigerator for cooling.

1 ml portions of the sample were transferred to 2 ml chromatography vials and 100 μl of perfluoromethylcyclopentane (b.p. 49.5° C.) was added to each vial. The vials were shaken on an Espe CapMix® for 75 seconds and the samples were immediately cooled on ice. The contents of the vials were collected in a larger vial, and the emulsion was fractionated to remove excess lipid and larger emulsion droplets. The sample was then characterised with respect to size distribution and total particle volume concentration using a Coulter counter; the median droplet size was 2-4 μm, confirming that the emulsion was acceptable for injection. The particle volume concentration measurement was used to adjust the concentration to about 10 μl/ml disperse phase using 10 mM Tris buffer solution. The emulsion was stored in a refrigerator until use.

The positively charged perfluoromethylcyclopropane emulsion is administrated intravenously in amounts corresponding to 0.04 μl perfluoromethylcyclopropane/kg body weight.

Preparation 3

An amount of the perfluorobutane gas dispersion from Preparation 1 corresponding to 0.1 μl gas/kg body weight was diluted in a 10% sucrose solution to a total volume of 2.5 ml, and filled into an injection syringe. An amount of preparation 2 corresponding to 0.04 μl perfluoromethylcyclopentane/kg body weight was diluted in a 10% sucrose solution to a total volume of 2.5 ml, and filled into another injection syringe. The content of both syringes was injected intravenously and simultaneously via a T-tube connector and a common cannula. The injection was performed in 5 seconds, and the cannula and tubing was flushed with some 5 ml of isotonic saline after the injection.

General Procedure for In Vivo Imaging of Dog Heart

A 20 kg dog was anaesthetised and mechanically ventilated, a mid-line sternotomy was performed, and the anterior pericardium was removed. Mid-line short-axis B-mode imaging of the heart was performed through a low-attenuating 30 mm silicone rubber spacer, using an ATL HDI-5000 scanner equipped with a P3-2 transducer. The frame rate was 21 Hz and the mechanical index was 0.8. Myocardial contrast was evaluated pre-dose (baseline) and 1½ min after injection (peak), and also at other time points when specified.

### EXAMPLE 1

### Imaging During Normal Myocardial Blood Flow

a) Imaging Using Preparation 3 During Continuous Room Air Ventilation

Preparation 3 was injected into the dog during continuous room air ventilation (partial pressure ratio of O₂/N₂=21/79 at normal atmospheric pressure)). The resulting myocardial contrast effect was intense and peak contrast was observed around 1½ min after injection. The myocardial contrast had returned to baseline levels approximately 10 minutes after injection.

b) Imaging Using Preparation 3 During Continuous Ventilation with Oxygen/Helium

Procedure of Example 1(a) was repeated except that helium and oxygen were mixed to attain ventilation with a partial pressure ratio of O₂/He of 21/79. An equilibration time of 10 minutes was allowed before injection of Preparation 3. The resulting myocardial contrast effect was comparable to Example 1(a).

c) Imaging Using Preparation 3 During Continuous Ventilation with Various Partial Pressures of Inert Gas

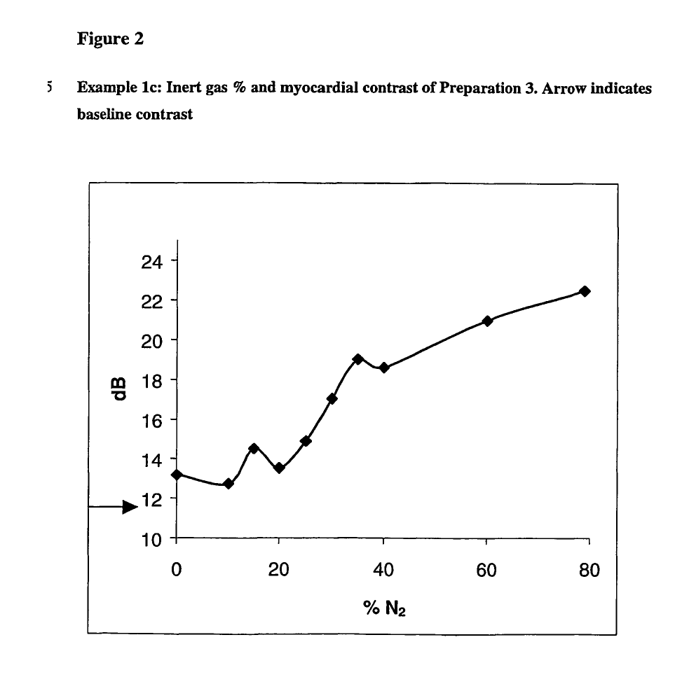

Procedure of Example 1 (a) was repeated except that oxygen and room air was mixed to attain partial pressure ratios of O₂/N₂ of 100/0, 90/10, 85/15, 80/20, 75/25, 70/30, 65/35, 60/40, 40/60 and 21/79. After each adjustment of the partial pressure ratios of O₂/N₂, an equilibration time of 5 minutes was allowed before injection. The resulting myocardial contrast effect was absent at partial pressure of nitrogen below 20 kPa and increased gradually as partial pressure of nitrogen rose above 20 kPa, attaining the same contrast effect level as observed in Example 1(a) when partial pressure ratio of O₂/N₂ was 21/79 (FIG. 2). When present the myocardial contrast duration was decreased during ventilation with decreasing partial pressure of nitrogen.

d) Imaging Using Preparation 3 During Room Air Ventilation Followed by a Change to Ventilation with a Gas Devoid of Inert Gas

Procedure of Example 1(a) was repeated except that the partial pressure ratios of O₂/N₂ were changed from 21/79 to 100/0 at 1 minute after injection. The resulting myocardial contrast effect with peak at 1½ min was identical to Example 1 (a), but was more short-lived and had returned to baseline levels approximately 4 minutes after injection.

e) Imaging Using Preparation 3 During Oxygen/Helium Ventilation Followed by a Change to Oxygen Ventilation

Procedure of Example 1(b) was repeated except that the ventilation with partial pressure ratio of O₂/He of 21/79 was changed to ventilation with O₂/He of 100/0 at 1½ minute after injection. The resulting myocardial contrast effect was identical to Example 1(d).

f) Imaging Using Preparation 3 During Ventilation with Oxygen, Followed by a Change to Room Air Ventilation

Procedure of Example 1(a) was repeated except that the partial pressure ratios of O₂/N₂ were changed from 100/0 to 21/79 at 0, 30 and 60 seconds before injection. An equilibration time of 5 min during O₂/N₂ 100/0 ventilation was allowed before injection. When partial pressure ratio of O₂/N₂ was changed at injection, the resulting myocardial contrast effect was absent, as also seen during continuous ventilation with 100/0 partial pressure ratio of O₂/N₂ observed in Example 1(c). When partial pressure ratio of O₂/N₂ was changed 30 seconds before injection, the resulting myocardial contrast effect was slightly less than observed in Example 1(a). When partial pressure ratio of O₂/N₂ was changed 60 seconds before injection, the resulting myocardial contrast effect was comparable to Example 1(a).

### EXAMPLE 2

### Imaging During Myocardial Reactive Hyperaemia

Procedures of “General Procedure” above were repeated except that a snare was placed around the proximal part of the LAD (left anterior descending coronary artery) and a flow transducer was placed distal to the snare. Tightening of the snare caused complete occlusion of the LAD, followed by reactive hyperaemia and increased LAD flow when the snare was released.

a) Imaging Using Preparation 3 During Ventilation with Oxygen, Followed by Room Air Ventilation

An equilibration period of 10 min during ventilation with a partial pressure ratio of O₂/N₂ 100/0 preceded the LAD occlusion. LAD was completely occluded from 75 to 5 seconds before injection and partial pressure ratio of O₂/N₂ was changed from 100/0 to 21/79 at 15 seconds before injection of Preparation 3. The LAD flow increased after release of the LAD snare, to a peak of 5 times the pre-occlusion level 5 seconds after injection, and then rapidly (within 40 seconds after injection) decreased to a level corresponding to 1.4 times the LAD flow before occlusion, at which level it was stable for more than 3 minutes after release. Peak contrast was observed around 1½ min after injection. The resulting myocardial contrast effect in the hyperaemic myocardium was intense and comparable to or slightly higher than observed in Example 1(a). The resulting myocardial contrast effect in the normal myocardium was only slightly above baseline. The difference in myocardial contrast between hyperaemic and normal myocardium was significantly greater than observed in control experiments with continuous room air breathing.

b) Imaging Using Preparation 3 During Ventilation with Oxygen, Followed by a Change to Helium/Oxygen Ventilation

Procedure of Example 2(a) was repeated except that the ventilation was changed from a partial pressure ratio of O₂/He of 100/0 to 21/79. The resulting myocardial contrast effect in the hyperaemic and normal myocardium was comparable to Example 2(a).

### EXAMPLE 3

### Imaging During Reduced Myocardial Blood Flow

Procedures of “General Procedure” were repeated except that a snare was placed around the proximal part of the LAD and a flow transducer was placed distal to the snare. Tightening the snare caused a controllable LAD flow reduction by partial LAD occlusion.

a) Imaging Using Preparation 3 During Continuous Room Air Ventilation

LAD flow was reduced to 50% of baseline by tightening the snare and Preparation 3 was injected into the dog. Peak contrast was observed around 1½ min after injection. The resulting myocardial contrast effect in the myocardium with normal blood flow was comparable to Example 1(a). The resulting myocardial contrast effect in the myocardium supplied by the occluded LAD was approximately 6 dB below the contrast in the myocardium with normal blood flow.

b) Imaging Using Preparation 3 During Ventilation with Oxygen, Followed by Room Air Ventilation

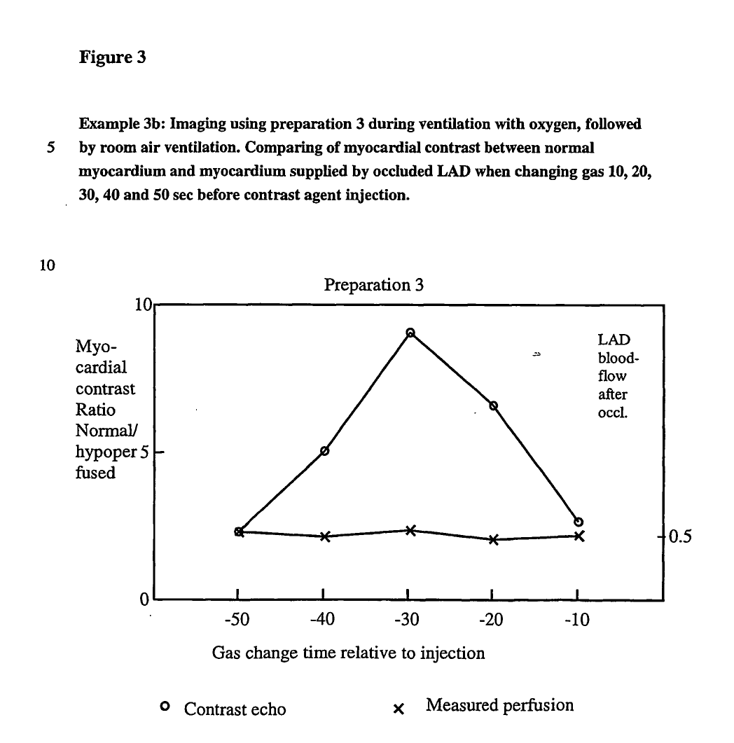

Procedure of Example 3(a) was repeated except that an equilibration period of 5 min during ventilation with a partial pressure ratio of O₂/N₂ of 100/0 preceded the injections, and partial pressure ratio of O₂/N₂ was changed to 21/79 at 10, 20, 30, 40 and 50 seconds before injection. Peak contrast was observed around 1½ min after injection. The resulting myocardial contrast effect in the myocardium with normal blood flow was comparable to or higher than Example 1(a) when the inspiratory gases were changed 20, 30, 40 and 50 sec before injection. When the inspiratory gas was changed 10 sec before injection, the resulting myocardial contrast effect in the myocardium with normal blood flow was less than in Example 1(a). The resulting myocardial contrast effect in the myocardium supplied by the occluded LAD was slightly above baseline when the inspiratory gases were changed 10, 20, 30 and 40 sec before injection. When the inspiratory gases were changed 50 sec before injection, the resulting myocardial contrast effect in the myocardium supplied by the occluded LAD was slightly below Example 1(a). The difference in myocardial contrast between normal myocardium and myocardium supplied by occluded LAD was comparable to the difference observed in Example 3(a) when the inspiratory gases where changed 10 and 50 sec before injection. When the inspiratory gas was changed 20, 30 and 40 sec before injection, the contrast difference was markedly higher, with a peak of 12 dB at 30 sec. Please see FIG. 3.

### EXAMPLE 4

### Imaging During Dobutamine Stress and Partial Coronary Occlusion

Procedures of “General Procedure” were repeated except that a snare was placed around the proximal part of the LAD and a flow transducer was placed distal to the snare. Tightening the snare caused a controllable LAD flow reduction by partial LAD occlusion. Infusion of dobutamine (20 μg/kg/min) increased the coronary blood flow to about twice of baseline values, while the LAD flow was maintained a baseline levels and thus about 50% less than the other parts of the coronary circulation.

a) Imaging Using Preparation 3 During Ventilation with Oxygen, Followed by Room Air Ventilation

After initiation of dobutamine infusion and stabilisation of coronary flow, LAD flow was reduced by 50%. An equilibration period of 10 min during ventilation with a partial pressure ratio of O₂/N₂ of 100/0 preceded the injection of Preparation 3 into the dog. Partial pressure ratio of O₂/N₂ was changed to 21/79 at 30 seconds before injection. The resulting myocardial contrast effect in the myocardium with non-occluded blood flow was comparable to or slightly higher than Example 1(a). Peak contrast was observed around 1½ min after injection. The resulting myocardial contrast effect in the myocardium supplied by the occluded LAD was only slightly above baseline. This regional difference in contrast effects is far above the effect observed during the same conditions and continuous room air breathing.

b) Imaging Using Preparation 3 During Ventilation with Room Air, Followed by Oxygen Ventilation

Procedure of Example 4(a) was repeated except that partial pressure ratio of O₂/N₂ was changed from 21/79 to 100/0 at 75 seconds after injection of Preparation 3. The resulting peak myocardial contrast effect in the myocardium with non-occluded blood flow was comparable to Example 1(a) and slightly above the contrast in the myocardium supplied by the occluded LAD. Peak contrast was observed around 1½ min after injection. The myocardial contrast was further observed 2½ and 3 minutes after dosing and the contrast effect in the myocardium supplied by the occluded LAD was transiently higher than the contrast effect in the myocardium with non-occluded blood flow, before returning to baseline levels.

### EXAMPLE 5

### Imaging Using Infusion/Bolus Combination of Preparation 3 During Ventilation with Oxygen Followed by Room Air

The procedure of Example 4 (a) is repeated, but the contrast agent is given as a slow i.v. infusion (0.05 μl gas kg⁻¹ min⁻¹ and 0.02 μl perfluoromethylcyclopentane kg⁻¹ min⁻¹), by a slow injection from the syringes described in preparation 3, starting 10 seconds before switching from oxygen to room air. The development of myocardial contrast effect in the normally perfused region of the myocardium is monitored continuously. Start of faint contrast effects in the normal myocardium is observed about 30 seconds after switching gases, and an i.v. bolus injection of (0.1 μl gas kg⁻¹ and 0.04 μl perfluoromethylcyclopentane kg⁻¹) is then immediately given from another pair of syringes according to preparation 3, and the infusion is stopped. The resulting contrast effects 60 seconds later is similar to Example 4 (a).

1. Method of ultrasound investigation of a human or non-human animal subject comprising
i) administering a gas-microbubble containing ultrasound contrast agent to said subject
ii) administering at least two gases or gas mixtures to said subject, said gases or gas mixtures having different partial pressure of inert gas, said gases or gas mixtures being administered either prior to, during and/or after the administration of said ultrasound contrast agent
iii) detecting ultrasound signals from said subject
iv) optionally, generating an image from said detected signals

2. Method as claimed in claim 1 wherein the investigation comprises an assessment of perfusion in tissues of said subject.

3. Method as claimed in claim 1 or 2 wherein said gases or gas mixtures are administrated by inhalation.

4. Method as claimed in any of claims 1-3 wherein said inert gas comprises any metabolically inert and biocompatible gas or mixture of gases; preferably nitrogen, helium, argon, other noble gases, N₂O, or mixtures thereof; most preferably nitrogen.

5. Method as claimed in any of claims 1-4 wherein the administration of a first gas or gas mixture is followed by the administering of a second gas or gas mixture, said second gas or gas mixture having a low partial pressure of inert gas when said first gas or gas mixture has a high pressure of inert gas, and having a high pressure of inert gas when said first gas or gas mixture has a low pressure of inert gas.

6. Method as claimed in claim 5 wherein said first gas or gas mixture has a low partial pressure of inert gas and wherein said second gas or gas mixture has a high partial pressure of inert gas.

7. Method as claimed in claims 5 or 6 wherein said first gas or gas mixture comprises oxygen and said second gas or gas mixture is room air.

8. Method as claimed in any of claims 5-7 wherein said high partial pressure of inert gas is between 75 and 85 kPa, preferably about 79 kPa and wherein said low partial pressure of inert gas is below 60 kPa, preferably below 40 kPa, more preferably below 20 kPa, most preferably below 5 kPa.

9. Method as claimed in any of claims 1-8 wherein said gas-microbubbles contain a gas of low water solubility, preferably fluorinated gases, more preferably fluorocarbons or sulfur hexafluoride, most preferably perfluoropropane, perfluorobutanes, perfluoropentanes and perfluorohexanes.

10. Method as claimed in any of claims 1-9 wherein said microbubbles are stabilised by a gas-stabilising material being selected from a coalescence-resistant surface membrane, preferred a filmogenic protein; a polymer material, preferred polylactic acid, polyglycolic acid, or copolymers of polylactic and polyglycolic acid; a non-polymeric and non-polymerisable wall-forming material; and a surfactant, preferably one or more phospholipids.

11. Method as claimed in any of claims 1-10 wherein said gas-microbubbles are capable of developing a change in echogenicity upon contact with tissue having a partial pressure of inert gas different from the partial pressure of inert gas inside said microbubbles.

12. Method as claimed in any of claims 1-11 wherein said gas-microbubbles have a prolonged transit time through the tissues providing a sufficient gas exchange with said tissues.

13. Method as claimed in claim 12 wherein said prolonged transit time of microbubbles through the tissues is caused by at least transient increase in size of said microbubbles or by tissue-specific vectors located on the surface material of said microbubbles.

14. Method as claimed in claim 12 wherein said prolonged transit time of microbubbles is caused by the contrast agent being a combined preparation comprising a stabilised dispersed gas and a co-administered composition comprising a volatile component capable of evaporation in vivo into the dispersed gas so as at least transiently to increase the size of the microbubbles.

15. Method as claimed in any of claims 1-14 wherein administration of said gases or gas mixtures are controllably timed with the administration of said contrast agent.

16. Method as claimed in claim 15 wherein the administering of a first gas or gas mixture is followed by the administration of a second gas or gas mixture, and wherein the administration of said second gas is starting before, during or after administration of said ultrasound contrast agent.

17. Method as claimed in claim 16 wherein administration of said first gas or gas mixture has a duration of at least 5 minutes, preferably of at least 10 minutes.

18. Method as claimed in any of claims 16 or 17 wherein said first gas or gas mixture has a low partial pressure of inert gas and administering of said second gas or gas mixture is started 90 to 0 seconds before administering of said contrast agent, preferably 60 to 15 seconds before, and most preferably about 30 seconds before start of administering of the contrast agent.

19. Method as claimed in any of claims 1-18 wherein the contrast agent is administered by infusion followed by a bolus injection of the contrast agent.

20. Use of a gas-microbubble containing ultrasound contrast agent and at least two gases having different partial pressure of inert gas for the manufacturing of an ultrasound imaging agent for detecting ultrasound signals from a subject.

21. Use of an ultrasound imaging agent as claimed in claim 20 for use in assessing perfusion in tissue of a subject.

22. Use of an ultrasound imaging agent as claimed in claim 20 or 21 wherein said contrast agent is a combined preparation for simultaneous, separate or sequential use as a contrast agent in ultrasound imaging, said preparation comprising:
i) a first composition which is an injectable aqueous medium comprising dispersed gas microbubbles; and
ii) a second composition which is an injectable oil-in-water emulsion wherein the oil phase comprises droplets of a volatile component capable of evaporation in vivo into said dispersed gas microbubbles so as at least transiently to increase the size thereof.

23. Ultrasound imaging agent comprising a gas-microbubble containing ultrasound contrast agent and at least two gases or gas mixtures having different partial pressure of inert gas.

24. Kit comprising a gas-microbubble containing ultrasound contrast agent and at least two gases or gas mixtures having different partial pressure of inert gas.
